SEQUENCE LISTING (2 sequences)

SEQ ID NO: 1 - RNA, 23 nt, Artificial Sequence
  Nucleotide sequence of a first nucleic acid
aaggagcuuacaaucuagcuggg

SEQ ID NO: 2 - RNA, 23 nt, Artificial Sequence
  Nucleotide sequence of a second nucleic acid
cccagcuagauuguaagcuccuu

(19) United States
(12) Patent Application Publication
(10) Pub. No.: US 2020/0370045 A1
(43) Pub. Date: Nov. 26, 2020

(54) METHOD OF TREATING TYPE II DIABETES AND OBESITY AND METHOD OF SCREENING A MEDICAMENT FOR THE SAME

(71) Applicant: Tsinghua University, Beijing (CN)

(72) Inventors: Peng LI, Beijing (CN); Qiqi LI, Beijing (CN)

(21) Appl. No.: 16/766,330

(22) PCT Filed: Dec. 21, 2017
(86) PCT No.: PCT/CN2017/117832

§ 371 Date: May 22, 2020

(30) Foreign Application Priority Data
Nov. 23, 2017 (CN) 201711185446.6

(51) Int. Cl. C12N 15/113; A61K 31/365; A61K 31/426; G01N 33/50; C12N 15/11; C12N 9/22; A61K 38/46; A61K 31/7088

(52) U.S. Cl. CPC C12N 15/113; C12N 2800/80; A61K 31/426; G01N 33/5044; G01N 33/5088; C12N 15/11; C12N 9/22; A61K 38/465; A61K 31/7088; C12N 2310/14; C12N 2310/122; C12N 2310/11; C12N 2310/128; C12N 2310/20; A61K 31/365

(57) ABSTRACT

The present invention provides a use of an agent for preparing a medicament for inhibiting microRNA-708. The medicament is used for at least one of the following: reducing intracellular triglyceride content, inhibiting differentiation of fat cells, resisting obesity, promoting insulin sensitivity, increasing respiratory metabolic rates, increasing energy consumption, increasing the number of mitochondria, up-regulating oxidative phosphorylation or heat-producing genes, relieving insulin resistance, resisting fatty liver and treating or preventing Type 2 diabetes.

### PRIORITY

The application claims a priority to and benefits of Chinese Patent Applications No. 201711185446.6, filed with the National Intellectual Property Administration, PRC on Nov. 23, 2017, the entire content of which is incorporated herein by reference.

### FIELD

The present disclosure relates to the field of biology, in particular, to the use of a reagent in preparing a medicament, a method screening a medicament, and a pharmaceutical composition.

### BACKGROUND

MicroRNA (miRNA) is endogenous small RNAs in a length of about 20-24 nucleotides, providing various important regulatory functions in cells. Each miRNA may have multiple target genes, while several miRNAs may regulate the same gene. This complex regulatory network can not only regulate the expression levels of multiple genes through one miRNA, but also finely regulate the expression level of a certain gene through combination of several miRNAs. It is speculated that miRNAs regulate one-third of human genes. Recent studies have shown that approximately 70% of mammalian miRNAs are located in the transcription units (TUs) region (Rodriguez et al, 2004), and most of them are located in the intron region (Kim & Nam, 2006). The positions of some intron miRNAs are highly conserved among different species. miRNAs are not only conserved for the gene position, but also show high homology in sequence (Pasquinelli et al, 2000; Ruvkun et al, 2001; Lee & Ambros, 2001). The conservation of miRNAs is closely related to its important function. As miRNAs are closely related to its target gene evolution, it is helpful to further investigate its mechanism and function by studying such evolution.

MicroRNAs play a crucial role in regulating many aspects of life activities, but the relationship between microRNAs and obesity/diabetes remains to be explored and discovered by scientists.

### SUMMARY

This disclosure is accomplished based on the inventor's discovery and knowledge of the following facts and problems.

Two mouse models well-established in Laboratory are used by the inventors: ob (with leptin knockout) mice and ob/fsp27^{-/-} (with leptin and fsp27 both knockout) mice, exhibiting obesity and lean phenotypes, respectively. Abdominal fats are collected from respective two mice models for RNA extraction, miRNA microarray and statistical analysis, real-time PCR and functional verification, demonstrating that microRNA-708 significantly increases intracellular triglycerides.

To this end, in one aspect, the present disclosure provides in embodiments use of a reagent for microRNA-708 inhibition in manufacture of a medicament for use in at least one of decreasing an intracellular triglyceride level, suppressing adipocyte differentiation, defending against obesity, enhancing insulin sensitivity, increasing a respiratory-metabolic rate, increasing energy expenditure, increasing the number of mitochondria, up-regulating an oxidative phosphorylation- or thermogenesis-related gene, relieving insulin resistance, defending against fatty liver, and treating or preventing type II diabetes. It is found by the present inventors that use of the medicament manufactured with the microRNA-708 inhibiting reagent can effectively decrease an intracellular triglyceride level, suppress adipocyte differentiation, defend against obesity, enhance insulin sensitivity, increase a respiratory-metabolic rate, increase energy expenditure, increase the number of mitochondria, up-regulate an oxidative phosphorylation- or thermogenesis-related gene, relieve insulin resistance, treat or prevent type II diabetes and defend against fatty liver.

According to embodiments of the present disclosure, the above-mentioned use may further include at least one of the following additional technical features.

According to some embodiments of the present disclosure, the inhibition is achieved by at least one of short hairpin RNA (shRNA), antisense nucleic acid, ribozyme, dominant negative mutation, CRISPR-Cas9, CRISPR-Cpf1 and zinc finger nuclease, thereby effectively achieving microRNA-708 knockout/knockdown or inhibiting microRNA-708 function.

According to some embodiments of the present disclosure, the inhibition is achieved by short hairpin RNA (shRNA), the reagent includes a first nucleic acid including a nucleotide sequence set forth in SEQ ID NO: 1.

(SEQ ID NO: 1)
AAGGAGCUUACAAUCUAGCUGGG.

The first nucleic acid having the above nucleotide sequence can effectively achieve microRNA-708 knockout/knockdown, thereby effectively inhibiting microRNA-708 function. It is found by the present inventors that the microRNA-708 knockout mice can resist high fat diets-induced obesity, with insulin sensitivity improved significantly, the respiratory-metabolic rate and the energy expenditure increased remarkably, subcutaneous white adipose tissue brown obviously, the number of mitochondria increased apparently, and the oxidative phosphorylation- or thermogenesis-related gene up-regulated strikingly.

According to some embodiments of the present disclosure, the inhibition is achieved by an antisense nucleic acid, and the reagent includes a second nucleic acid including a nucleotide sequence set forth in SEQ ID NO: 2.

(SEQ ID NO: 2)
CCCAGCUAGACUUGUAAGUCCUU.

The second nucleic acid having the above nucleotide sequence can effectively inhibit microRNA-708 function. It is found by the present inventors that the obese mice treated with antisense nucleic acid are distinctly declined in terms of obesity levels and significantly alleviated for insulin resistance symptoms.

According to some embodiments of the present disclosure, the obesity is high fat diets-induced obesity. The microRNA-708 inhibiting reagent has better inhibitory effects on the high fat diets-induced obesity.

According to some embodiments of the present disclosure, the oxidative phosphorylation- or thermogenesis-related gene includes at least one of ucpl, cidea, pgcla, ppara and Dio2. It is found by the present inventors that the oxidative phosphorylation- or thermogenesis-related gene is up-regulated strikingly in the microRNA-708 knockout mice, suggesting that microRNA-708 can suppress the expression level of the oxidative phosphorylation- or thermogenesis-related gene, thereby inhibiting the respiratory-metabolic rate and the energy expenditure.

In another aspect, the present disclosure provides in embodiments a method of screening a medicament for use in at least one of decreasing an intracellular triglyceride level, suppressing adipocyte differentiation, defending against obesity, enhancing insulin sensitivity, increasing a respiratory-metabolic rate, increasing energy expenditure, increasing the number of mitochondria, up-regulating an oxidative phosphorylation- or thermogenesis-related gene, relieving insulin resistance, defending against fatty liver, and treating or preventing type II diabetes. According to embodiments of the present disclosure, the method includes contacting a candidate medicament with a disease model, and microRNA-708 inhibition in the disease model after the contact is an indicator of the candidate medicament being a target medicament. With the screening method according to embodiments of the present disclosure, the target medicament thus obtained can be used in decreasing an intracellular triglyceride level, suppressing adipocyte differentiation, defending against obesity, enhancing insulin sensitivity, increasing a respiratory-metabolic rate, increasing energy expenditure, increasing the number of mitochondria, up-regulating an oxidative phosphorylation- or thermogenesis-related gene, relieving insulin resistance, defending against fatty liver, and treating or preventing type II diabetes.

According to embodiments of the present disclosure, the above method may further include at least one of the following additional technical features.

According to embodiments of the present disclosure, the microRNA-708 inhibition includes down-regulation of microRNA-708 expression or inhibition of microRNA-708 function, such that the regulatory function of microRNA-708 on the oxidative phosphorylation- or thermogenesis-related gene is effectively suppressed.

According to embodiments of the present disclosure, the disease model is adipocytes or a mouse obesity model. It is found by the present inventors that the mirRNA-708 is highly and specifically expressed in adipose tissue from obese human (relative to that of healthy human), and the adipocyte or the mouse obesity model is selected to effectively magnify the inhibitory effect if the candidate medicament is able to inhibit mirRNA-708, such that the target medicament is obtained with high reliability.

In a further aspect, the present disclosure provides in embodiments a pharmaceutical composition. According to embodiments of the present disclosure, the pharmaceutical composition includes the reagent as defined above. The pharmaceutical composition according to embodiments of the present disclosure may be effectively used in decreasing an intracellular triglyceride level, suppressing adipocyte differentiation, defending against obesity, enhancing insulin sensitivity, increasing a respiratory-metabolic rate, increasing energy expenditure, increasing the number of mitochondria, up-regulating an oxidative phosphorylation- or thermogenesis-related gene, relieving insulin resistance, defending against fatty liver, and treating or preventing type II diabetes.

According to embodiments of the present disclosure, the above pharmaceutical composition may further include at least one of the following additional technical features.

According to embodiments of the present disclosure, the pharmaceutical composition may further include an additional reagent for use in treating or preventing type II diabetes or obesity. In some specific embodiments, the additional reagent includes at least one selected from orlistat, Thiazolidinedione (TZD), and the like. In other embodiments, the present pharmaceutical composition may further include fillers, anticoagulants, lubricants, wetting agents, aromatics and preservatives.

In a still further aspect, the present disclosure provides in embodiments a method of treating or preventing type II diabetes or obesity. According to embodiments, the method includes administering the pharmaceutical composition as defined above to a patient.

According to embodiments of the present disclosure, the present pharmaceutical composition can decrease an intracellular triglyceride level, suppress adipocyte differentiation, defend against obesity, enhance insulin sensitivity, increase a respiratory-metabolic rate, increase energy expenditure, increase the number of mitochondria, up-regulate an oxidative phosphorylation- or thermogenesis-related gene, relieve insulin resistance, and defend against fatty liver, such that the present pharmaceutical composition can be administered when treating or preventing type II diabetes or obesity.

As used herein, the term “administering” refers to introducing a predetermined amount of a substance into a patient in a suitable manner. The present pharmaceutical composition can be administered by any common route as long as it can reach the intended tissue. Various modes of administration are contemplated, including peritoneum, vein, muscle, subcutaneous, cortex, oral, topical, nasal, lung, and rectum, which are not limited herein to these exemplified modes of administration. Preferably, the present pharmaceutical composition can be administered as an injection preparation. In addition, the present pharmaceutical composition can be administered using a specific device that delivers the active ingredient to target cells.

The administration frequency and dosage of the present pharmaceutical composition can be determined by various relevant factors, including the type of disease to be treated, the dosing regimen, the patient's age, sex, body weight and severity of the disease, and the type of the medicament as the active ingredient. According to some embodiments of the present disclosure, the daily dose can be divided into one, two or more doses in a suitable form for administration in one, two or more times over the entire period, as long as the therapeutically effective amount is reached.

The term “therapeutically effective amount” refers to an amount of compound sufficient to significantly improve certain symptoms associated with a disease or condition, that is, an amount that provides a therapeutic effect for a given condition and dosing regimen. For example, in the treatment of type II diabetes, a drug or compound that reduces, prevents, delays, inhibits, or blocks any symptoms of a disease or disorder should be therapeutically effective. A therapeutically effective amount of a drug or compound is not necessary to cure the disease or condition, but will provide treatment for the disease or condition so that the onset of the individual's disease or condition is delayed, prevented or prevented, or the symptoms of the disease or condition are alleviated, or the duration of the disease or condition is changed, or for example, the disease or condition becomes less severe, or recovery is accelerated.

The term “treating” is used to refer to obtaining the desired pharmacological and/or physiological effect. The effect may be preventive in terms of completely or partially preventing the disease or its symptoms, and/or may be therapeutic in terms of partially or completely curing the disease and/or the adverse effects caused by the disease. “treating” as used herein covers treating a disease (mainly type II diabetes and obesity) in mammals, especially human, including:

(a) preventing occurrence of a disease (e.g., type II diabetes) or condition in an individual who are susceptible to the disease but have not been diagnosed with the disease; (b) inhibiting a disease, such as retarding development of the disease; or (c) relieving a disease, such as alleviating the symptoms associated with the disease. “Treating” as used herein encompasses any administration of a drug or compound to an individual to treat, cure, alleviate, ameliorate, alleviate or inhibit the individual's disease, including but not limited to administering the pharmaceutical composition described herein to an individual in need thereof.

According to embodiments of the present disclosure, the present pharmaceutical composition may be used in combination with conventional treatment methods and/or therapies, or may be used separately with conventional treatment methods and/or therapies. When the present pharmaceutical composition is administered in combination with the additional reagent, they can be administered to an individual sequentially or simultaneously. Alternatively, the present pharmaceutical composition may include the present reagent or a pharmaceutically acceptable excipient in combination with an additional therapeutic or prophylactic agent known in the art.

### BRIEF DESCRIPTION OF THE DRAWINGS

FIG. 1 is a graph showing significant increased intracellular triglycerides by mirRNA-708 according to an embodiment of the present disclosure.

FIG. 2 is a graph showing specific high expression of mirRNA-708 in adipose tissue according to an embodiment of the present disclosure.

FIG. 3 is a graph showing increasing mirRNA-708 expression levels as adipocytes differentiate according to an embodiment of the present disclosure.

FIG. 4 is a graph showing obvious lower weight gain of the mirRNA-708 knockout mouse model fed with high fat diets relative to that of healthy mice according to an embodiment of the present disclosure.

FIG. 5 is a graph showing obvious lower fat mass of the mirRNA-708 knockout mouse model fed with high fat diets relative to that of healthy mice according to an embodiment of the present disclosure.

FIG. 6 is a graph showing obvious higher glucose tolerance of the mirRNA-708 knockout mice relative to that of healthy mice according to an embodiment of the present disclosure.

FIG. 7 is a graph showing obvious higher insulin tolerance of the mirRNA-708 knockout mice relative to that of healthy mice according to an embodiment of the present disclosure.

FIG. 8 is a graph showing significant higher respiratory-metabolic rate and energy expenditure of the mirRNA-708 knockout mice relative to that of healthy mice according to an embodiment of the present disclosure.

FIG. 9 is a graph showing obvious browning of subcutaneous white adipose tissue of the mirRNA-708 knockout mice according to an embodiment of the present disclosure.

FIG. 10 is a graph showing strikingly up-regulated expression levels of oxidative phosphorylation- and thermogenesis-related genes of the mirRNA-708 knockout mice according to an embodiment of the present disclosure.

FIG. 11 is a graph showing significantly relieved fatty liver of the mirRNA-708 knockout mice fed with high fat diets relative to that of healthy mice according to an embodiment of the present disclosure.

FIG. 12 is a graph showing significantly reduced hepatic lipid content of the mirRNA-708 knockout mice fed with high fat diets relative to that of healthy mice according to an embodiment of the present disclosure.

FIG. 13 is a picture showing obvious reduced obesity levels of obese mouse treated with antisense nucleic acids of mirRNA-708 according to an embodiment of the present disclosure.

FIG. 14 is a graph showing obvious relieved glucose tolerance of obese mice treated with antisense nucleic acids of mirRNA-708 according to an embodiment of the present disclosure.

FIG. 15 is a graph showing obvious relieved insulin resistance of obese mice treated with antisense nucleic acids of mirRNA-708 according to an embodiment of the present disclosure.

FIG. 16 is a graph showing obvious higher expression levels of mirRNA-708 in adipose tissue of obese human relative to that of healthy human according to an embodiment of the present disclosure.

### DETAILED DESCRIPTION

Reference will be made in detail to embodiments of the present disclosure, and examples of the embodiments are shown in the drawings. The embodiments described herein with reference to drawings are explanatory, illustrative, and used to generally understand the present disclosure. The embodiments shall not be construed to limit the present disclosure.

### EXAMPLE 1

Significantly Increased Intracellular Triglyceride Caused by microRNA-708

Mouse primary fat cells were cultured for 48 hours in the presence of mirRNA-708 transfection (a well-established sequence synthesized in Shanghai GenePharma Co., Ltd) or scramble transfection (negative control) with lipofectamine 2000. After washed with PBS 2-3 times and transferred in 2 ml PBS to a 15 ml falcon tube, the cells were added with 8 ml (Hexanelsoproponal=3:2) for vigorous vibration on a shaker overnight. After about 10 min still standing, when white precipitation in the middle layer disappeared, the organic phase at the upper layer was transferred to a glass tube, which was heated at 70° C. at the bottom and blow-dried with nitrogen at the top, and then dissolved in 100 μl toluene. The resulting protein-containing aqueous solution at the bottom was centrifuged at 4000 rpm for 20 min, with the aqueous solution discarded and pellets dried at 60° C. to be powders (by placed on a heat block in an ultra-clean bench), which was then dissolved in 1 ml 0.2M KOH overnight. In the next day, the protein concentrations were assayed and used as internal reference. The collected test sample and the standard lipid solution were subjected to TLC (Hexane:Ether:Acetic acid=70:30:1), followed by immersing in copper sulfate for about 20 seconds, and then slightly blow-drying till no obvious droplets existed. After dried at 100 to 120° C. for 5 to 10 min, gel imaging system was applied for quantitative analysis.

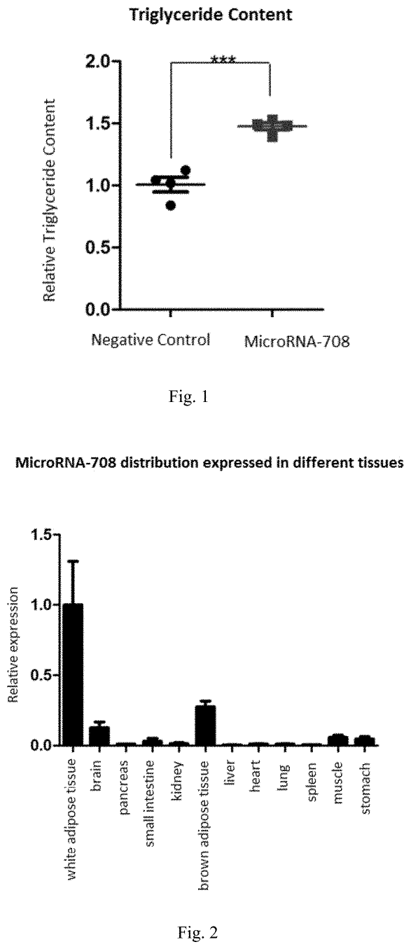

It is found by the present inventors that the intracellular triglyceride is significantly increased by microRNA-708 as shown in FIG. 1.

### EXAMPLE 2

Role of microRNA-708 in Adipose Metabolism

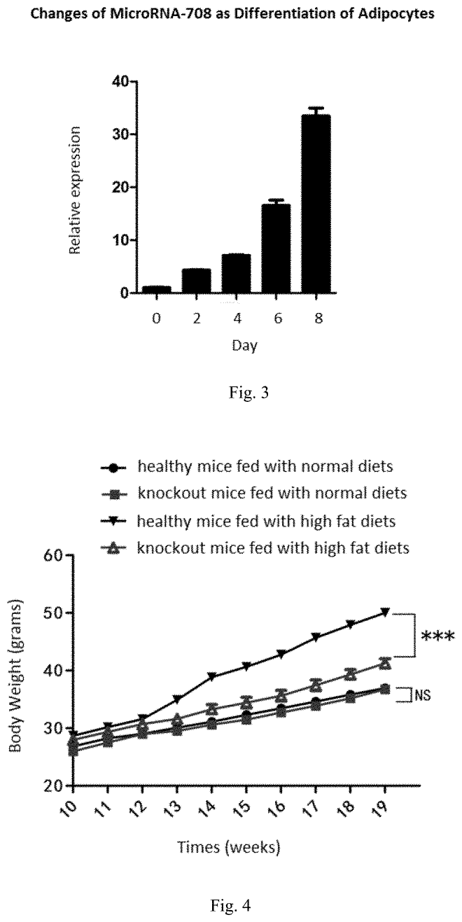

Total RNA was extracted from various tissues of mice respectively for detection of microRNA-708 distribution by Realtime PCR, from which it is found by the present inventors that microRNA-708 is specifically and highly expressed in adipose tissue. Further, the mouse primary preadipocyte isolated by centrifugation were induced to differentiate to mature adipocytes in vitro, during which increasing microRNA-708 expression levels were observed as adipocytes differentiated, proving that microRNA-708 is closely related to the function of adipose tissue or adipocytes. The results are shown in FIGS. 2 and 3.

After 10 weeks' accommodation, healthy and microRNA-708 knockout mice (purchased from Nanjing Institute of Model Biology and achieved by cas9 engineering technology) were fed with normal diets (ND) and the high fat diets (HFD) respectively for another 10 weeks, with body weight measured weekly. As shown in FIGS. 4 and 5, obvious lower weight gain was observed in the microRNA-708 knockout mice fed with HFD relative to that of healthy mice, indicating that the microRNA-708 knockout mice can defend against HFD-induced obesity.

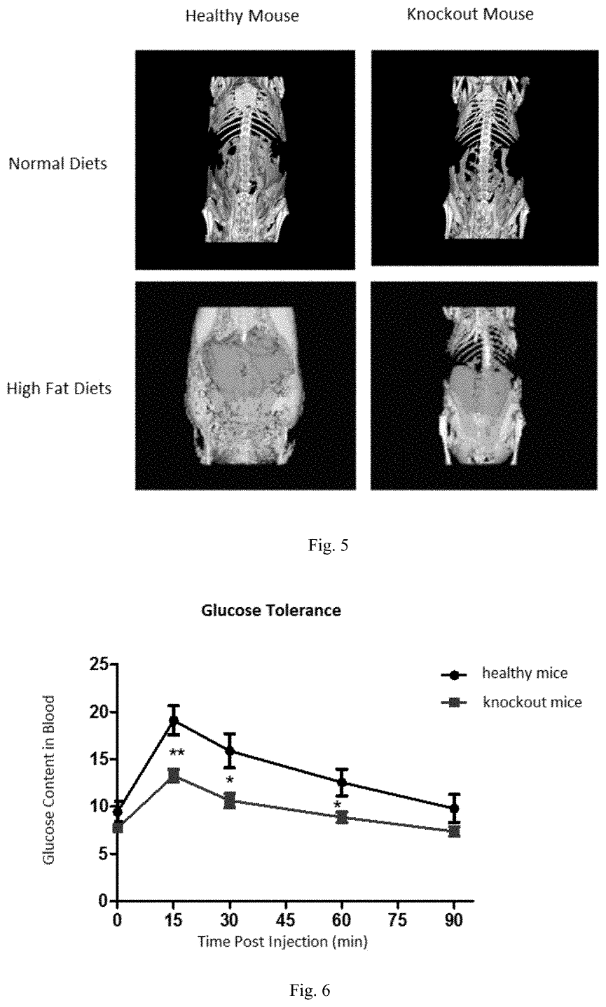

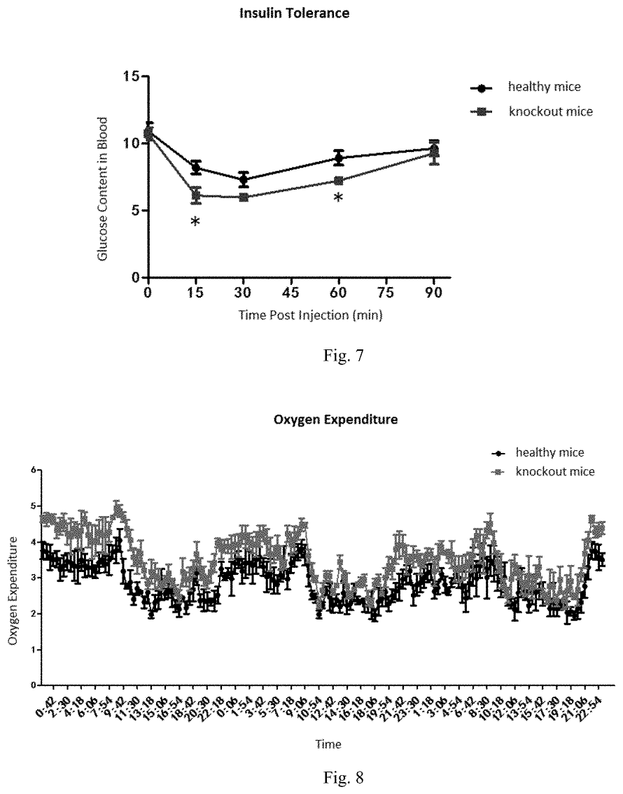

As obesity is closely related to type II diabetes, the present inventors also examined the role of microRNA-708 in insulin sensitivity, where insulin tolerance test (ITT) and glucose tolerance test (GTT) were adopted. ITTs of the microRNA-708 knockout mice were assayed 0, 15, 30, 60, and 90 min after an intraperitoneal injection of insulin (diluted as 0.5 U/ml in saline and dosed at 0.5-1.2 U insulin per kg body mass, e.g., 0.5 U insulin per kg body mass, 0.027 U/10 g=2.7 U/kg) following a 4 h fast. GTTs of the microRNA-708 knockout mice were assayed 0, 15, 30, 60, and 90 min after an intraperitoneal injection of glucose (diluted as 20% glucose solution in saline and dosed at 0.5-2 g glucose per kg body mass, e.g., 1 g glucose per kg body mass) following overnight fast. As shown in FIGS. 6 and 7, obvious increased insulin sensitivity were observed in the microRNA-708 knockout mice fed with ND as compared with the healthy mice, suggesting that knockout or knockdown microRNA-708 effectively improved insulin sensitivity, a promising therapeutic target for type II diabetes.

Furthermore, oxygen expenditure and carbon dioxide emissions per minute per body mass of healthy and microRNA-708 knockout mice were monitored individually in the metabolic chamber. As shown in FIG. 8, obvious higher respiratory-metabolic rates were observed in the microRNA-708 knockout mice as compared to the healthy mice, presumably the reason for defending obesity.

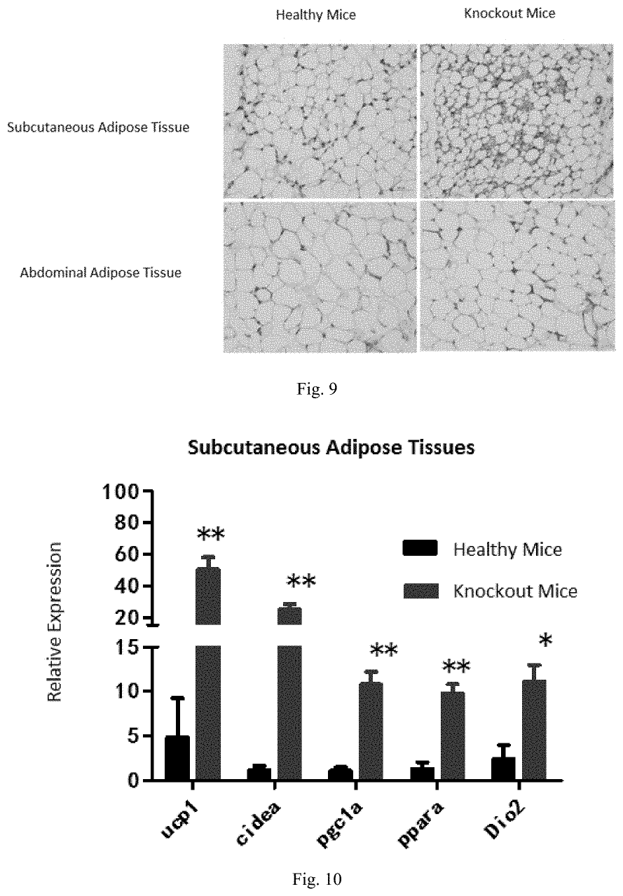

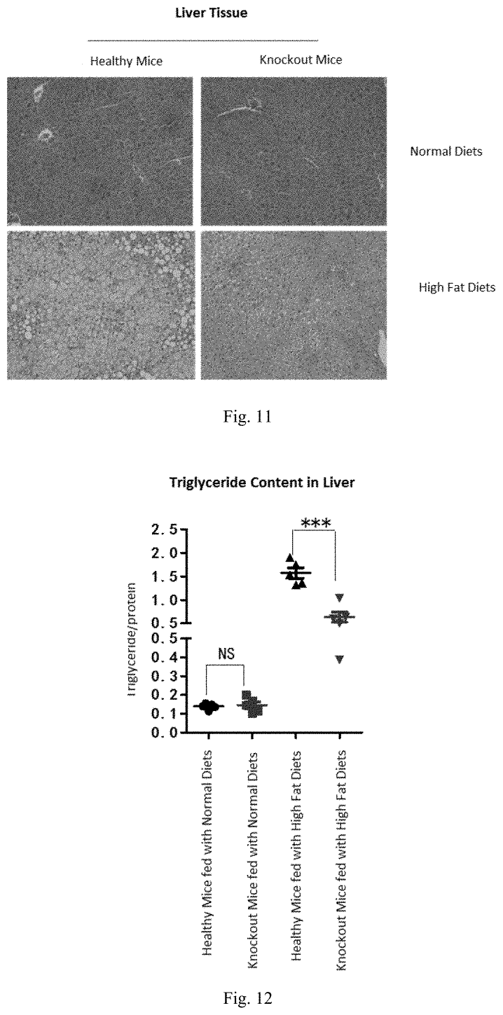

The subcutaneous adipose tissue from healthy and microRNA-708 knockout mice were fixed in 10% paraformaldehyde and stained with haematoxylin-eosin for electron microscope analysis. As shown in FIG. 9, obvious browning of subcutaneous white adipose tissue was observed in the mirRNA-708 knockout mice relative to the healthy mice, where lipid droplets were changed from larger unilocular to smaller multilocular cells, and the number of mitochondria was increased significantly. In FIG. 11, significantly relieved fatty liver of the mirRNA-708 knockout mice fed with high fat diets as compared to that of healthy mice.

After extracted from healthy and microRNA-708 knockout mice, respective RNA was subjected to reverse transcription to obtain cDNA for gene detection by Realtime-PCR. FIG. 10 demonstrated that the oxidative phosphorylation- or thermogenesis-related genes were up-regulated strikingly.

Next, the lipid contents were investigated between healthy and microRNA-708 knockout mice. By means of TLC detection, the significantly reduced hepatic triglyceride content was observed in the mirRNA-708 knockout mice fed with high fat diets relative to that of healthy mice, as shown in FIG. 12.

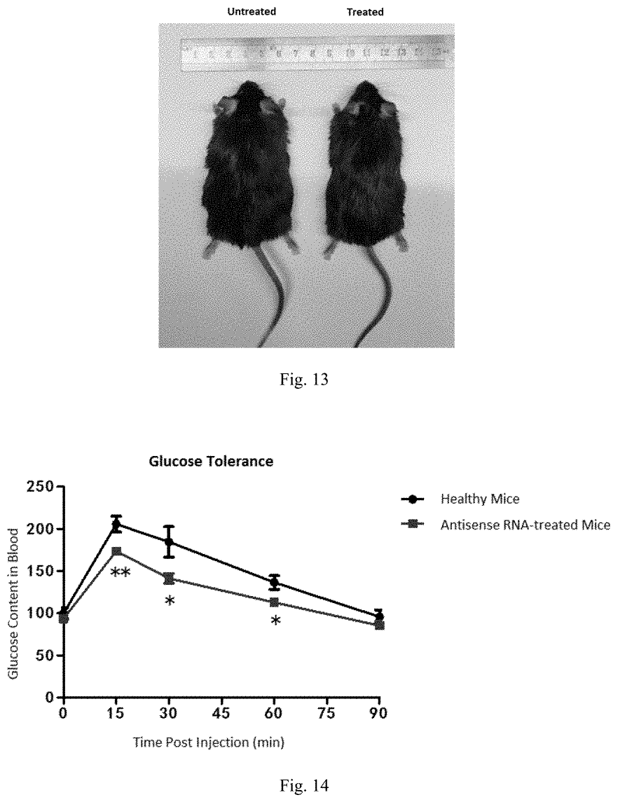

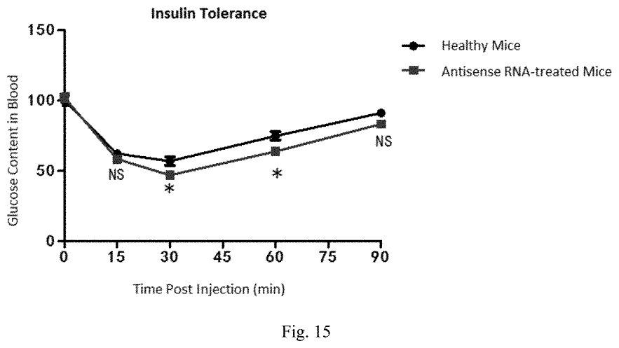

A designed RNA sequence (CCCAGCUAGACUUGUAAGUCCUU (SEQ ID NO: 2)), reverse complementary to microRNA-708, was subcutaneously injected at 10 D/g to 2-month old obese mice (fed with HFD for two months) weekly for four weeks. By such treatment, the obesity levels in the obese mice were declined distinctly and insulin tolerance symptoms were also significantly alleviated, shown in FIGS. 13 to 15.

### EXAMPLE 3

microRNA-708 Profile in the Human Obesity Model

The present inventors collected fat tissues from different regions of healthy and obese patients (including abdominal adipose tissue, abdominal subcutaneous adipose tissue and inguinal subcutaneous adipose tissue) from the Department of Endocrinology, Shanghai Sixth Hospital. These tissues were homogenized for extraction of total RNA by TRIZOL. The steps are as follows:

1. adding Trizol to cells or tissues, still standing at room temperature for 5 min for sufficient lysis;

2. centrifuging at 12,000 rpm for 5 min with pellet discarded;

3. adding 200 μl chloroform per 1 ml Trizol, vortex for mixing and placing at room temperature for 15 min;

4. centrifuging at 12,000 g for 15 min at 4° C.;

5. pipetting the upper aqueous phase into another centrifuge tube;

6. adding 0.5 ml isopropanol per 1 ml Trizol, vortex for mixing, placing at room temperature for 5 to 10 min;

7. centrifuging at 12,000 g for 10 min at 4° C. with supernatant discarded and thus RNA pellet obtained;

8. adding 1 ml 75% ethanol per 1 ml Trizol, gently shaking to re-suspend the pellet;

9. centrifuging at 8,000 g for 5 min at 4° C. with supernatant discarded;

10. air-drying at room temperature or vacuum drying for 5 to 10 min.

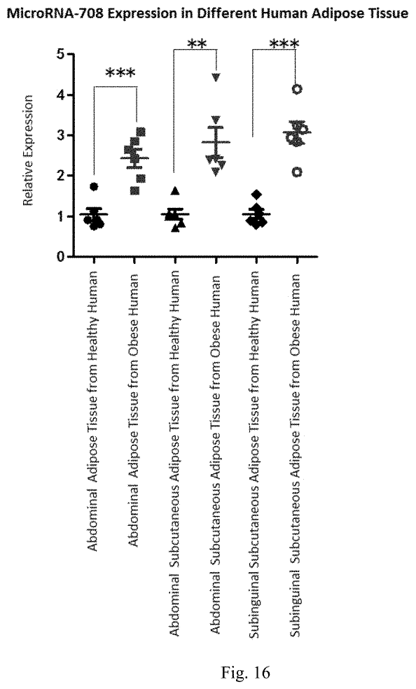

After reverse transcription was performed using miRNA reverse transcription kit from TransGen Biotech, the expression level of mirRNA-708 in these tissues were detected by Real-Time PCT. It is found that microRNA was expressed significantly higher in adipose tissues of obese human relative to that of healthy human, as shown in FIG. 16.

Reference throughout this specification to “an embodiment”, “some embodiments”, “one embodiment”, “another example”, “an example”, “a specific example” or “some examples” means that a particular feature, structure, material, or characteristic described in connection with the embodiment or example is included in at least one embodiment or example of the present disclosure. Thus, the appearances of the phrases such as “in some embodiments”, “in one embodiment”, “in an embodiment”, “in another example”, “in an example”, “in a specific example” or “in some examples” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those ordinarily skilled in the art that the above embodiments cannot be construed to limit the present disclosure, and changes, alternatives, and modifications can be made in the embodiments in the scope of the present disclosure.

1.-13. (canceled)

14. A method of treating type II diabetes or obesity, comprising introducing to a subject in need thereof a therapeutically effective amount of a reagent for microRNA-708 inhibition.

15. The method according to claim 14, wherein the inhibition is achieved by at least one of short hairpin RNA (shRNA), an antisense nucleic acid, ribozyme, dominant negative mutation, CRISPR-Cas9, CRISPR-Cpfl and zinc finger nuclease.

16. The method according to claim 14, wherein the inhibition is achieved by knocking down or knocking out a first nucleic acid comprising a nucleotide sequence set forth in SEQ ID NO: 1.

17. The method according to claim 14, wherein the inhibition is achieved by introducing a second nucleic acid comprising a nucleotide sequence set forth in SEQ ID NO: 2, wherein the second nucleic acid is reverse complementary to the first nucleic acid.

18. The method according to claim 14, wherein the obesity is induced by high fat diets.

19. The method according to claim 14, wherein the reagent for microRNA-708 inhibition is capable of decreasing an intracellular triglyceride level.

20. The method according to claim 14, wherein the reagent for microRNA-708 inhibition is capable of enhancing insulin sensitivity.

21. The method according to claim 20, wherein the reagent for microRNA-708 inhibition is capable of improving insulin tolerance.

22. The method according to claim 20, wherein the reagent for microRNA-708 inhibition is capable of improving glucose tolerance.

23. The method according to claim 14, wherein the reagent for microRNA-708 inhibition is capable of increasing an energy expenditure rate for microRNA-708 inhibition.

24. The method according to claim 23, wherein the reagent for microRNA-708 inhibition is capable of increasing a respiratory-metabolic rate for microRNA-708 inhibition.

25. The method according to claim 23, wherein the reagent for microRNA-708 inhibition is capable of increasing the number of mitochondria.

26. The method according to claim 23, wherein the reagent for microRNA-708 inhibition is capable of up-regulating an oxidative phosphorylation- or thermogenesis-related gene.

27. The method according to claim 26, wherein the oxidative phosphorylation- or thermogenesis-related gene comprises at least one of ucpl, cidea, pgcla, ppara and Dio2.

28. The method according to claim 14, wherein the reagent for microRNA-708 inhibition is capable of alleviating fatty liver.

29. The method according to claim 14, wherein the reagent for microRNA-708 inhibition is introduced to the subject sequentially or simultaneously in combination with an additional reagent for type II diabetes and obesity.

30. The method according to claim 29, wherein the additional reagent comprises at least one selected from orlistat and thiazolidinedione.

31. A method of screening a medicament for type II diabetes and obesity, comprising
contacting a candidate medicament with a disease model,
wherein microRNA-708 inhibition in the disease model after the contact is an indicator of the candidate medicament being a target medicament.

32. The method according to claim 31, wherein the disease model is adipocytes or a mouse obesity model.

33. The method according to claim 31, wherein the microRNA-708 inhibition comprises down-regulation of microRNA-708 expression or inhibition of microRNA-708 function.
